(19) United States
(12) Patent Application Publication
(10) Pub. No.: US 2009/0222093 A1
(43) Pub. Date: Sep. 3, 2009

(54) Nucleus Implant and Method of Installing Same

(75) Inventors: Mingyan Liu, Bourg la Reine (FR); Jean-Charles LeHuec, Pessac (FR); Kenneth J. Burkus, Columbus, GA (US); Loic Josse, Denens (FR)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/039,519

(22) Filed: Feb. 28, 2008

(51) Int. Cl. A61F 2/44

(52) U.S. Cl. 623/17.12; 623/17.16

(57) ABSTRACT

A nucleus implant is disclosed. The nucleus implant can be configured to be installed within an intervertebral disc between an inferior vertebra and a superior vertebra. Further, the nucleus implant can include a solid core and an expandable chamber that can be disposed at least partially around the solid core. The expandable chamber can be expanded from a deflated position to an inflated position.

### FIELD OF THE DISCLOSURE

The present disclosure relates generally to orthopedics and spinal surgery. More specifically, the present disclosure relates to nucleus implants.

### BACKGROUND

In human anatomy, the spine is a generally flexible column that can take tensile and compressive loads. The spine also allows bending motion and provides a place of attachment for ribs, muscles and ligaments. Generally, the spine is divided into three sections: the cervical spine, the thoracic spine and the lumbar spine. The sections of the spine are made up of individual bones called vertebrae. Also, the vertebrae are separated by intervertebral discs, which are situated between adjacent vertebrae.

The intervertebral discs function as shock absorbers and as joints. Further, the intervertebral discs can absorb the compressive and tensile loads to which the spinal column may be subjected. At the same time, the intervertebral discs can allow adjacent vertebral bodies to move relative to each other a limited amount, particularly during bending, or flexure, of the spine. Thus, the intervertebral discs are under constant muscular and/or gravitational pressure and generally, the intervertebral discs are the first parts of the lumbar spine to show signs of “wear and tear”.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration

typically have occurred. For example, the altered mechanics of the facet joints and/or intervertebral disc may cause spinal stenosis, degenerative spondylolisthesis, and degenerative scoliosis.

One surgical procedure for treating these conditions is spinal arthrodesis, i.e., spine fusion, which can be performed anteriorally, posteriorally, and/or laterally. The posterior procedures include in-situ fusion, posterior lateral instrumented fusion, transforaminal lumbar interbody fusion (“TLIF”) and posterior lumbar interbody fusion (“PLIF”). Solidly fusing a spinal segment to eliminate any motion at that level may alleviate the immediate symptoms, but for some patients maintaining motion may be beneficial. It is also known to surgically replace a degenerative disc or facet joint with an artificial disc or an artificial facet joint, respectively. Additionally, it is known to surgically remove nucleus pulposus material from within an intervertebral disc and replace the nucleus pulposus material with an artificial nucleus.

### BRIEF DESCRIPTION OF THE DRAWINGS

FIG. 1 is a lateral view of a portion of a vertebral column;

FIG. 2 is a lateral view of a pair of adjacent vertrebrae;

FIG. 3 is a top plan view of a vertebra;

FIG. 4 is a cross section view of an intervertebral disc;

FIG. 5 is a plan view of a first embodiment of a nucleus implant;

FIG. 6 is another plan view of the first embodiment of the nucleus implant;

FIG. 7 is a cross-section view of the first embodiment of the nucleus implant taken along line 7-7 in FIG. 6;

FIG. 8 is a plan view of a second embodiment of a nucleus implant;

FIG. 9 is another plan view of the second embodiment of the nucleus implant;

FIG. 10 is a cross-section view of the second embodiment of the nucleus implant taken along line 10-10 in FIG. 9;

FIG. 11 is a plan view of a third embodiment of a nucleus implant;

FIG. 12 is another plan view of the third embodiment of the nucleus implant;

FIG. 13 is a cross-section view of the third embodiment of the nucleus implant taken along line 13-13 in FIG. 12;

FIG. 14 is a plan view of a fourth embodiment of a nucleus implant;

FIG. 15 is another plan view of the fourth embodiment of the nucleus implant;

FIG. 16 is a cross-section view of the fourth embodiment of the nucleus implant taken along line 16-16 in FIG. 15; and

FIG. 17 is a plan view of a fifth embodiment of a nucleus implant;

FIG. 18 is another plan view of the fifth embodiment of the nucleus implant;

FIG. 19 is a cross-section view of the fifth embodiment of the nucleus implant taken along line 19-19 in FIG. 18; and

FIG. 20 is a flow chart of a method of installing a nucleus implant;

### DETAILED DESCRIPTION OF THE DRAWINGS

A nucleus implant is disclosed. The nucleus implant can be configured to be installed within an intervertebral disc between an inferior vertebra and a superior vertebra. Further, the nucleus implant can include a solid core and an expandable chamber that can be disposed at least partially around the solid core. The expandable chamber can be expanded from a deflated position to an inflated position.

It will be noted that the chamber that is at least partially arranged around the core enables, when it is inflated, accurate positioning of the core. This implant provides mobility from one vertebra to another vertebra (rotation/flexion). The solid core of the implant makes easy the insertion of the latter in an intervertebral disc. Further, the solid core enables the implant to have certain features/properties (such as hardness) before the expansion of the chamber. This is because it is rather difficult to obtain these features/properties only when inflating the core and the chamber.

In another embodiment, a nucleus implant is disclosed. The nucleus implant can be configured to be installed within an intervertebral disc between an inferior vertebra and a superior vertebra. The nucleus implant can include a solid core that can include an outer surface. Also, the nucleus implant can include a toroid shaped expandable chamber that can be disposed at least partially around the solid core. The toroid shaped expandable chamber can include an inner surface and an outer surface. Further, the inner surface of the toroid shaped expandable chamber can engage the outer surface of the solid core and the outer surface of the toroid shaped expandable chamber can engage an annulus fibrosus of an intervertebral disc.

In yet another embodiment a nucleus implant is disclosed. The nucleus implant can be configured to be installed within an intervertebral disc between an inferior vertebra and a superior vertebra. The nucleus implant can include a solid core that can include an outer surface. Moreover, the nucleus implant can include a first toroid shaped expandable chamber that can be disposed at least partially around the solid core. The first toroid shaped expandable chamber can include an inner surface and an outer surface. The inner surface of the first toroid shaped expandable chamber can engage the outer surface of the solid core. The nucleus implant can also include a second toroid shaped expandable chamber that can disposed at least partially around the first toroid shaped expandable chamber. The second toroid shaped expandable chamber can include an inner surface and an outer surface. Further, the inner surface of the second expandable chamber can engage the outer surface of the first toroid shaped expandable chamber. Also, the outer surface of the second toroid shaped expandable chamber can engage an annulus fibrosus of the intervertebral disc.

In still another embodiment, a nucleus implant is disclosed. The nucleus implant can be configured to be installed within an intervertebral disc between an inferior vertebra and a superior vertebra. Moreover, the nucleus implant can include a solid core that can include an outer surface. The nucleus implant can also include a bowl shaped expandable chamber that can be disposed at least partially around the solid core. The bowl shaped expandable chamber can include an inner surface and an outer surface. The inner surface of the bowl shaped expandable chamber is configured to engage the outer surface of the solid core and the outer surface of the bowl shaped expandable chamber can engage an annulus fibrosus of the intervertebral disc, the superior vertebra, the inferior vertebra, or a combination thereof.

In yet still another embodiment, a nucleus implant is disclosed. The nucleus implant can be configured to be installed within an intervertebral disc between an inferior vertebra and a superior vertebra. Additionally, the nucleus implant can include a solid core that can include an outer surface. The nucleus implant can also include a U shaped expandable chamber that can be disposed at least partially around the solid core. The U shaped expandable chamber can include a first surface and a second surface. The first surface of the U shaped expandable chamber can engage the outer surface of the solid core and the second surface of the U shaped expandable chamber can engage an annulus fibrosus of the intervertebral disc.

In another embodiment, a method of installing a nucleus implant within an intervertebral disc between an inferior vertebra and a superior vertebra of a patient is disclosed. The method can include implanting the nucleus implant within the intervertebral disc. The nucleus implant can include a solid core and an expandable chamber at least partially around the solid core. Further, the method can include inflating the expandable chamber around the solid core. The expandable chamber can include an outer surface that engages an annulus fibrosus of the intervertebral disc when the expandable chamber is inflated. Further, a hardness of the solid core is greater than or equal to a hardness of the expandable chamber.

### Description of Relevant Anatomy

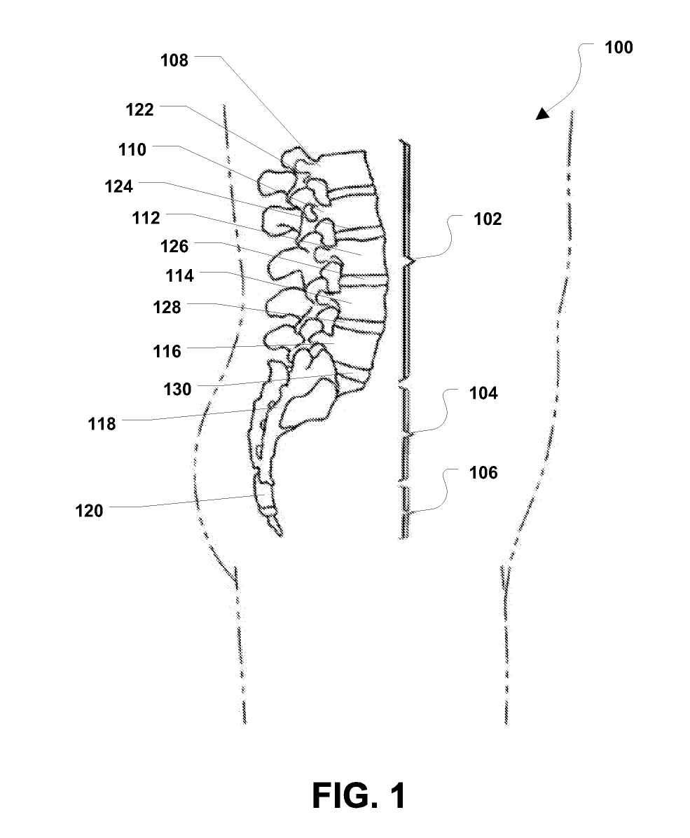

Referring initially to FIG. 1, a portion of a vertebral column, designated 100, is shown. As depicted, the vertebral column 100 includes a lumber region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 1, the lumbar region 102 includes a first lumber vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumber vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

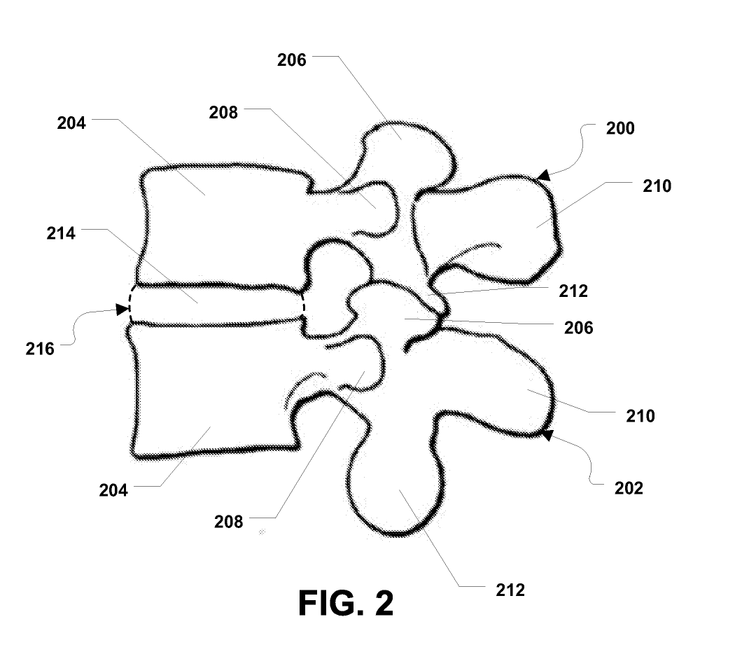

FIG. 2 depicts a detailed lateral view of two adjacent vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 2 further depicts an intervertebral space 214 that can be established between the superior vertebra 200 and the inferior vertebra 202 by removing an intervertebral disc 216 (shown in dashed lines).

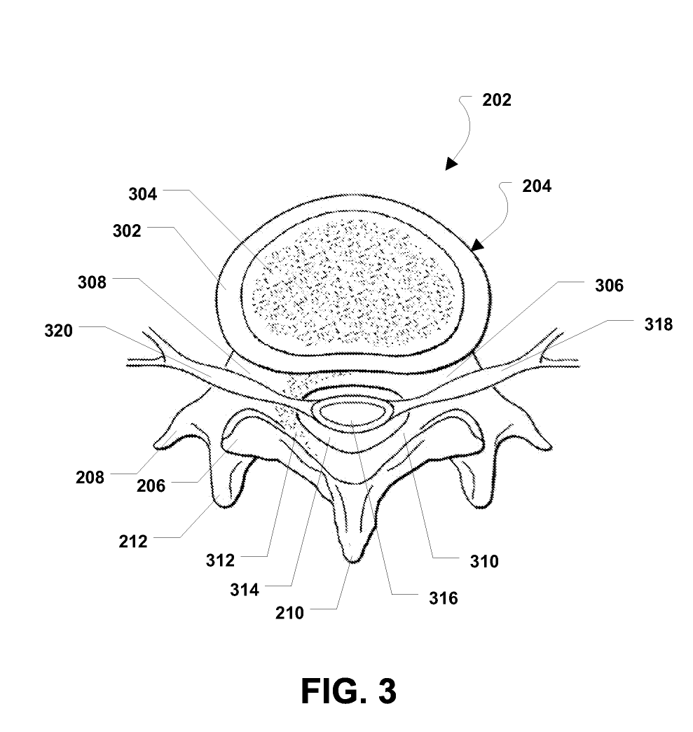

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer and weaker than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

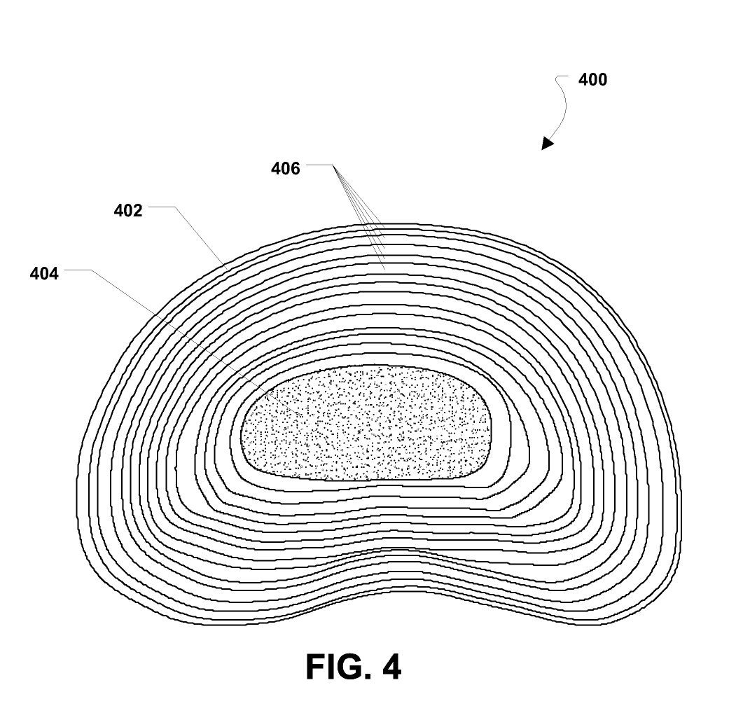

Referring now to FIG. 4, an intervertebral disc is shown and is generally designated 400. The intervertebral disc 400 is made up of two components: the annulus fibrosus 402 and the nucleus pulposus 404. The annulus fibrosus 402 is the outer portion of the intervertebral disc 400, and the annulus fibrosus 402 includes a plurality of lamellae 406. The lamellae 406 are layers of collagen and proteins. Each lamella 406 includes fibers that slant at 30-degree angles, and the fibers of each lamella 406 run in a direction opposite the adjacent layers. Accordingly, the annulus fibrosus 402 is a structure that is exceptionally strong, yet extremely flexible.

The nucleus pulposus 404 is the inner gel material that is surrounded by the annulus fibrosus 402. It makes up about forty percent (40%) of the intervertebral disc 400. Moreover, the nucleus pulposus 404 can be considered a ball-like gel that is contained within the lamellae 406. The nucleus pulposus 404 includes loose collagen fibers, water, and proteins. The water content of the nucleus pulposus 404 is about ninety percent (90%) at birth and decreases to about seventy percent (70%) by the fifth decade.

Injury or aging of the annulus fibrosus 402 may allow the nucleus pulposus 404 to be squeezed through the annulus fibers either partially, causing the disc to bulge, or completely, allowing the disc material to escape the intervertebral disc 400. The bulging disc or nucleus material may compress the nerves or spinal cord, causing pain. Accordingly, the nucleus pulposus 404 can be removed and replaced with an artificial nucleus.

### Description of a First Embodiment of a Nucleus Implant

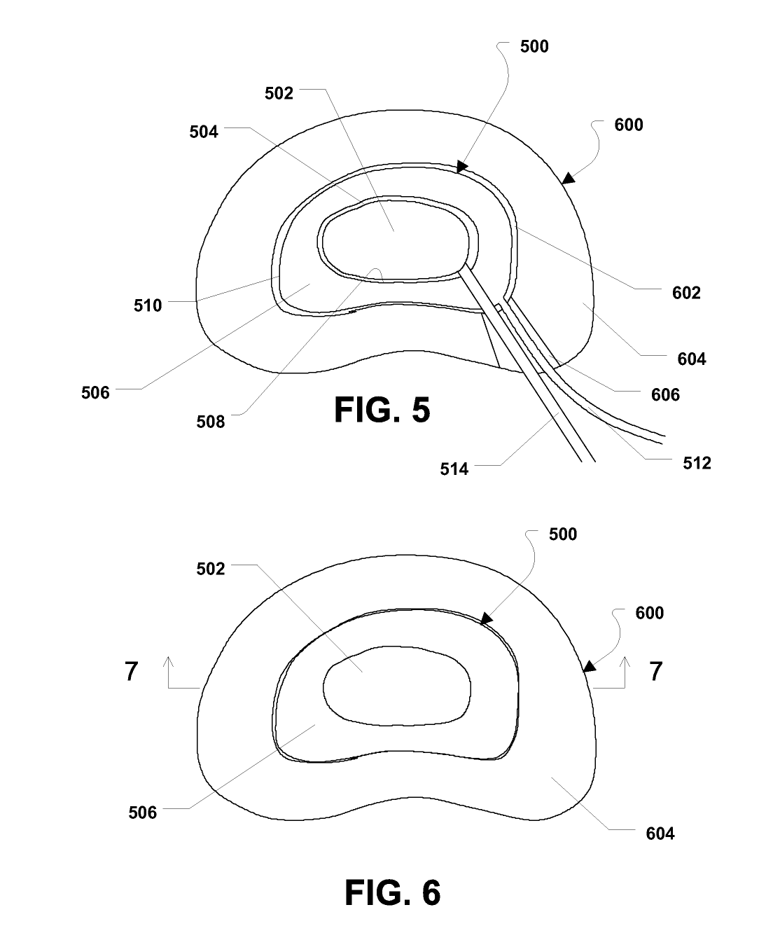

Referring to FIG. 5 through FIG. 7, an embodiment of a nucleus implant is shown and is designated 500. As shown, the nucleus implant 500 includes a solid core 502 that defines an outer surface 504. In a particular embodiment, the solid core 502 can have a cross-section that is generally elliptical. Alternatively, the solid core 502 can have a cross-section that is: generally circular, generally rectangular, generally square, generally triangular, generally trapezoidal, generally rhombic, generally quadrilateral, any generally polygonal shape, or any combination thereof.

As illustrated in FIG. 5 and FIG. 6, an expandable chamber 506 can be disposed around the solid core 502. In a particular embodiment, as shown, the expandable chamber 506 can have a generally toroidal shape. The shape of the chamber may enable, when expanded or inflated, the automatic positioning of the core. Further, the expandable chamber 506 can have a cross-section that is generally shaped like a kidney bean. Alternatively, the expandable chamber 506 can have a cross-section that is: generally elliptical, generally circular, generally rectangular, generally square, generally triangular, generally trapezoidal, generally rhombic, generally quadrilateral, any generally polygonal shape, or any combination thereof.

The expandable chamber 506 can define an inner surface 508 and an outer surface 510. In a particular embodiment, the inner surface 508 of the expandable chamber 506 can be attached to the outer surface 504 of the solid core 502. As such, proper placement of the expandable chamber 506 can be based on the placement of the solid core 502. Alternatively, the expandable chamber 506 can be separate from the solid core 502 and the expandable chamber 506 may engage the solid core 502 after the expandable chamber 506 is properly inflated. Alternately, the core and the chamber may be made of one and the same element, for example, for the sake of easiness.

As depicted in FIG. 5, the nucleus implant 500 can include an injection tube 512 that extends from the outer surface 510 of the expandable chamber 506. In a particular embodiment, the expandable chamber 506 of the nucleus implant 500 is expandable from a deflated position, shown in FIG. 5, to one selected position among a plurality of inflated positions, shown in FIG. 6, up to a maximum inflated position. Further, after the expandable chamber 506 is inflated, or otherwise expanded, the injection tube 512 can be removed, as depicted in FIG. 6.

Additionally, the nucleus implant 500 can include a core holder 514 that extends from the surface of the solid core 502. The core holder 514 can be used to position the nucleus implant 500 and hold the nucleus implant 500 in the proper position while the expandable chamber 506 is inflated. Moreover, the core holder 514 can be removed after the expandable chamber 506 is inflated. In a particular embodiment, the nucleus implant 500 can include a self-sealing valve (not shown) within the outer surface 510 of the expandable chamber 506, e.g., adjacent to the injection tube 512. The self-sealing valve can prevent the expandable chamber 506 from leaking material after the expandable chamber 506 is inflated and the injection tube 512 is removed.

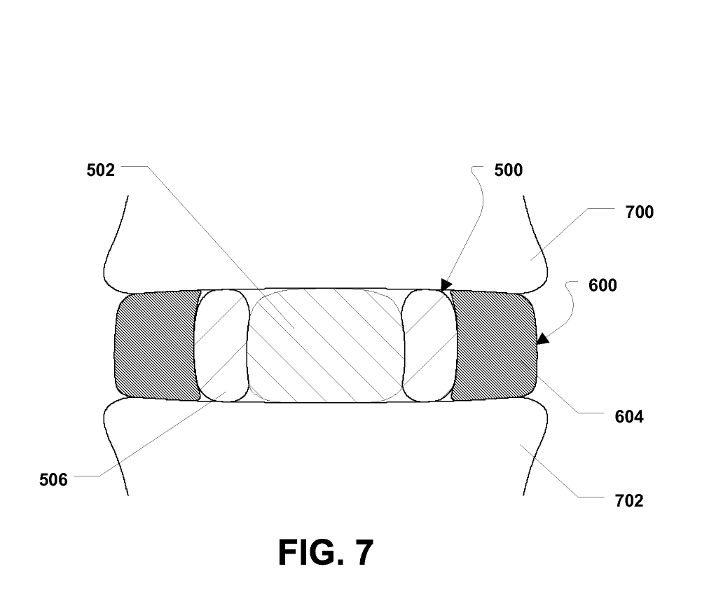

FIG. 7 indicates that the nucleus implant 500 can be implanted within an intervertebral disc 600 between a superior vertebra 700 and an inferior vertebra 702. More specifically, the nucleus implant 500 can be implanted within an intervertebral disc space 602 established within the annulus fibrosus 604 of the intervertebral disc 600. The intervertebral disc space 602 can be established by removing the nucleus pulposus (not shown) from within the annulus fibrosus 604.

In a particular embodiment, the expandable chamber 506 can be inflated so the inner surface 508 of the expandable chamber 506 engages the outer surface of the solid core 502 and the outer surface 510 of the expandable chamber 506 engages the annulus fibrosis 604. The nucleus implant 500 can provide shock-absorbing characteristics substantially similar to the shock absorbing characteristics provided by the nucleus pulposus. Further, in a particular embodiment, the hardness of the solid core 502 of the nucleus implant 500 is greater than or equal to the hardness of the material used to inflate the expandable chamber 506, i.e., after that material is cured. Additionally, the height of the solid core 502 can be greater than or equal to the height of the expandable chamber 506 when fully expanded. As shown in FIG. 7, the solid core 502 and the expandable chamber 506 of the nucleus implant 500 can be configured to provide proper support and spacing between the superior vertebra 700 and the inferior vertebra 702.

In a particular embodiment, the expandable chamber 506 of the nucleus implant 500 can be inflated with one or more injectable extended use approved medical materials that remain elastic after curing. Further, the injectable extended use approved medical materials can include polymer materials that remain elastic after curing.

For example, the polymer materials can include polyurethane materials, polyolefin materials, polyether materials, and silicone materials. Further, the polyolefin materials can include polypropylene, polyethylene, halogenated polyolefin, and flouropolyolefin. The polyether materials can include polyetherketone (PEK), polyetheretherketone (PEEK), polyetherketoneketone (PEKK), and polyaryletherketone (PAEK). Also, the silicone materials can include a silicone hydrogel.

In an alternative embodiment, the injectable extended use approved medical materials can include one or more fluids such as sterile water, saline, or sterile air. In alternative embodiments, the expandable chamber 506 of the nucleus implant 500 can be inflated with one or more of the following: fibroblasts, lipoblasts, chondroblasts, differentiated stem cells or other biologic factor which would create a motion limiting tissue when injected into a bioresorbable motion limiting scaffold.

In a particular embodiment, the nucleus implant 500 can be installed using a posterior surgical approach, as shown. Further, the nucleus implant 500 can be installed through a posterior incision 606 made within the annulus fibrosus 604 of the intervertebral disc 600. Alternatively, the nucleus implant 500 can be installed using an anterior surgical approach, a lateral surgical approach, or any other surgical approach well known in the art.

Forming a hole in the core of the implant may facilitate its manipulation. Further, such a hole may be used for introducing additional elements/other materials such as a temporary radiographic marker.

### Description of a Second Embodiment of a Nucleus Implant

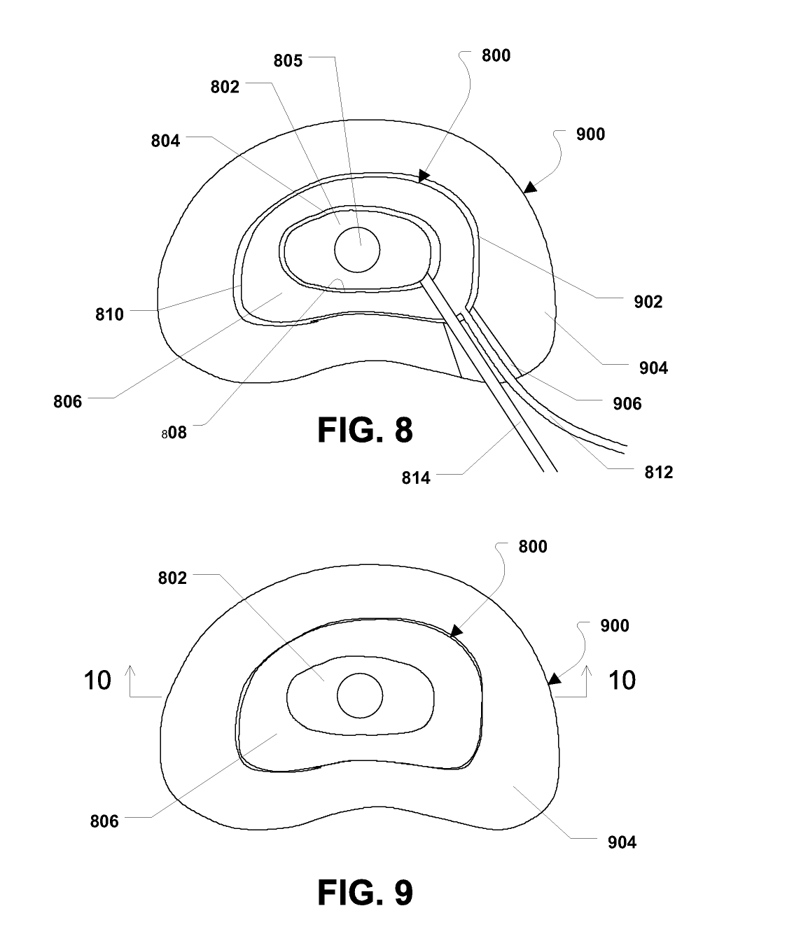

Referring to FIG. 8 through FIG. 10, an embodiment of a nucleus implant is shown and is designated 800. As shown, the nucleus implant 800 includes a solid core 802 that defines an outer surface 804. In a particular embodiment, the solid core 802 can have a cross-section that is generally elliptical. Alternatively, the solid core 802 can have a cross-section that is: generally circular, generally rectangular, generally square, generally triangular, generally trapezoidal, generally rhombic, generally quadrilateral, any generally polygonal shape, or any combination thereof.

Further, the solid core 802 of the nucleus implant 800 can be formed with a hole 805. In a particular embodiment, the hole 805 is formed in the center of the solid core 802. Moreover, the hole 805 can have a generally cylindrical shape. Alternatively, the hole 805 can have a generally prismatic shape. Moreover, the hole 805 can have a generally polyhedral shape.

As illustrated in FIG. 8 and FIG. 9, an expandable chamber 806 can be disposed around the solid core 802. In a particular embodiment, as shown, the expandable chamber 806 can have a generally toroidal shape. The shape of the chamber may enable, when expanded or inflated, the automatic positioning of the core. Further, the expandable chamber 806 can have a cross-section that is generally shaped like a kidney bean. Alternatively, the expandable chamber 806 can have a cross-section that is: generally elliptical, generally circular, generally rectangular, generally square, generally triangular, generally trapezoidal, generally rhombic, generally quadrilateral, any generally polygonal shape, or any combination thereof.

The expandable chamber 806 can define an inner surface 808 and an outer surface 810. In a particular embodiment, the inner surface 808 of the expandable chamber 806 can be attached to the outer surface 804 of the solid core 802. As such, proper placement of the expandable chamber 806 can be based on the placement of the solid core 802. Alternatively, the expandable chamber 806 can be separate from the solid core 802 and the expandable chamber 806 may engage the solid core 802 after the expandable chamber 806 is properly inflated.

As depicted in FIG. 8, the nucleus implant 800 can include an injection tube 812 that extends from the outer surface 810 of the expandable chamber 806. In a particular embodiment, the expandable chamber 806 of the nucleus implant 800 is expandable from a deflated position, shown in FIG. 8, to one selected position among a plurality of inflated positions, shown in FIG. 9, up to a maximum inflated position. Further, after the expandable chamber 806 is inflated, or otherwise expanded, the injection tube 812 can be removed, as depicted in FIG. 9.

Additionally, the nucleus implant 800 can include a core holder 814 that extends from the surface of the solid core 802. The core holder 814 can be used to position the nucleus implant 800 and hold the nucleus implant 800 in the proper position while the expandable chamber 806 is inflated. Moreover, the core holder 814 can be removed after the expandable chamber 806 is inflated. The toroidal shape of the chamber that is arranged around the core may enable accurate positioning of the core. In a particular embodiment, the nucleus implant 800 can include a self-sealing valve (not shown) within the outer surface 810 of the expandable chamber 806, e.g., adjacent to the injection tube 812. The self-sealing valve can prevent the expandable chamber 806 from leaking material after the expandable chamber 806 is inflated and the injection tube 812 is removed.

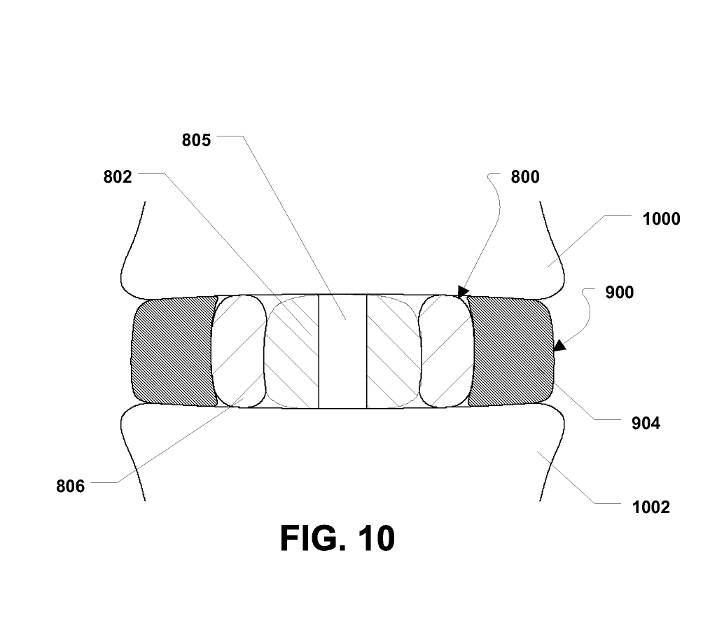

FIG. 10 indicates that the nucleus implant 800 can be implanted within an intervertebral disc 900 between a superior vertebra 1000 and an inferior vertebra 1002. More specifically, the nucleus implant 800 can be implanted within an intervertebral disc space 902 established within the annulus fibrosus 904 of the intervertebral disc 900. The intervertebral disc space 902 can be established by removing the nucleus pulposus (not shown) from within the annulus fibrosus 904.

In a particular embodiment, the expandable chamber 806 can be inflated so the inner surface 808 of the expandable chamber 806 engages the outer surface of the solid core 802 and the outer surface 810 of the expandable chamber 806 engages the annulus fibrosis 904. The nucleus implant 800 can provide shock-absorbing characteristics substantially similar to the shock absorbing characteristics provided by the nucleus pulposus. Further, in a particular embodiment, the hardness of the solid core 802 of the nucleus implant 800 is greater than or equal to the hardness of the material used to inflate the expandable chamber 806, i.e., after that material is cured. Additionally, the height of the solid core 802 can be greater than or equal to the height of the expandable chamber 806 when fully expanded. As shown in FIG. 10, the solid core 802 and the expandable chamber 806 of the nucleus implant 800 can be configured to provide proper support and spacing between the superior vertebra 1000 and the inferior vertebra 1002.

In a particular embodiment, the expandable chamber 806 of the nucleus implant 800 can be inflated with one or more injectable extended use approved medical materials that remain elastic after curing. Further, the injectable extended use approved medical materials can include polymer materials that remain elastic after curing.

For example, the polymer materials can include polyurethane materials, polyolefin materials, polyether materials, and silicone materials. Further, the polyolefin materials can include polypropylene, polyethylene, halogenated polyolefin, and flouropolyolefin. The polyether materials can include polyetherketone (PEK), polyetheretherketone (PEEK), polyetherketoneketone (PEKK), and polyaryletherketone (PAEK). Also, the silicone materials can include a silicone hydrogel.

In an alternative embodiment, the injectable extended use approved medical materials can include one or more fluids such as sterile water, saline, or sterile air. In alternative embodiments, the expandable chamber 806 of the nucleus implant 800 can be inflated with one or more of the following: fibroblasts, lipoblasts, chondroblasts, differentiated stem cells or other biologic factor which would create a motion limiting tissue when injected into a bioresorbable motion limiting scaffold.

In a particular embodiment, the nucleus implant 800 can be installed using a posterior surgical approach, as shown. Further, the nucleus implant 800 can be installed through a posterior incision 906 made within the annulus fibrosus 904 of the intervertebral disc 900. Alternatively, the nucleus implant 800 can be installed using an anterior surgical approach, a lateral surgical approach, or any other surgical approach well known in the art.

### Description of a Third Embodiment of a Nucleus Implant

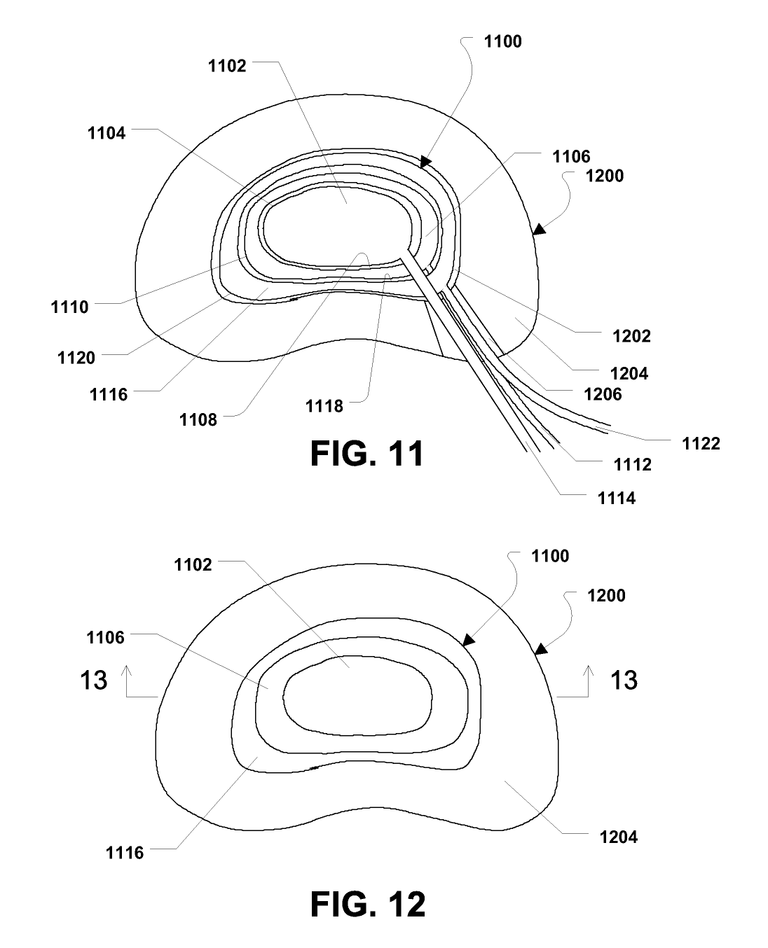

Referring to FIG. 11 through FIG. 13, a third embodiment of a nucleus implant is shown and is designated 1100. As shown, the nucleus implant 1100 includes a solid core 1102 that defines an outer surface 1104. In a particular embodiment, the solid core 1102 can have a cross-section that is generally elliptical. Alternatively, the solid core 1102 can have a cross-section that is: generally circular, generally rectangular, generally square, generally triangular, generally trapezoidal, generally rhombic, generally quadrilateral, any generally polygonal shape, or any combination thereof.

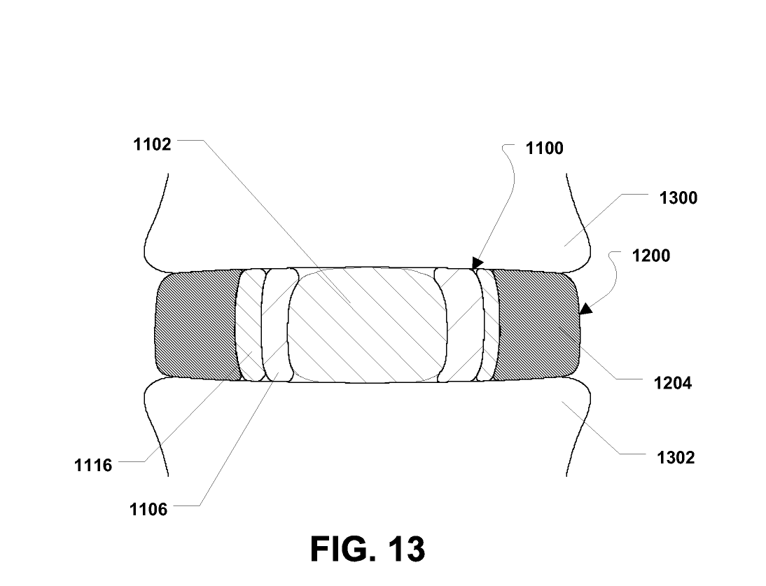

As illustrated in FIG. 11 and FIG. 12, a first expandable chamber 1106 can be disposed around the solid core 1102. In a particular embodiment, as shown, the first expandable chamber 1106 can have a generally toroidal shape. Further, as shown in FIG. 13, the first expandable chamber 1106 can have a cross-section that is generally shaped like a kidney bean. Alternatively, the first expandable chamber 1106 can have a cross-section that is: generally elliptical, generally circular, generally rectangular, generally square, generally triangular, generally trapezoidal, generally rhombic, generally quadrilateral, any generally polygonal shape, or any combination thereof.

The first expandable chamber 1106 can define an inner surface 1108 and an outer surface 1110. In a particular embodiment, the inner surface 1108 of the first expandable chamber 1106 can be attached to the outer surface 1104 of the solid core 1102. As such, proper placement of the first expandable chamber 1106 can be based on the placement of the solid core 1102. Alternatively, the first expandable chamber 1106 can be separate from the solid core 1102 and the first expandable chamber 1106 may engage the solid core 1102 after the first expandable chamber 1106 is properly inflated.

As depicted in FIG. 11, the nucleus implant 1100 includes a first injection tube 1112 that extends from the outer surface 1110 of the first expandable chamber 1106. In a particular embodiment, the first expandable chamber 1106 of the nucleus implant 1100 is expandable from a deflated position, shown in FIG. 11, to one selected position among a plurality of inflated positions, shown in FIG. 12, up to a maximum inflated position. Further, after the first expandable chamber 1106 is inflated, or otherwise expanded, the first injection tube 1112 can be removed, as depicted in FIG. 12.

FIG. 11 through FIG. 13 further show that the nucleus implant 1100 can include a second expandable chamber 1116 that can be disposed around the first expandable chamber 1106. In a particular embodiment, as shown, the second expandable chamber 1116 can have a generally toroidal shape. Further, as shown in FIG. 13, the second expandable chamber 1116 can have a cross-section that is generally shaped like a kidney bean. Alternatively, the second expandable chamber 1116 can have a cross-section that is: generally elliptical, generally circular, generally rectangular, generally square, generally triangular, generally trapezoidal, generally rhombic, generally quadrilateral, any generally polygonal shape, or any combination thereof.

The second expandable chamber 1116 can define an inner surface 1118 and an outer surface 1120. In a particular embodiment, the inner surface 1118 of the second expandable chamber 1116 can be attached to the outer surface 1110 of the first expandable chamber 1106 and the inner surface 1108 of the first expandable chamber 1106 can be attached to the outer surface 1104 of the solid core 1102. Alternatively, the second expandable chamber 1116 can be separate from the first expandable chamber 1106 and the solid core 1102. In such a configuration, the second expandable chamber 1116 can engage the first expandable chamber 1106 after the first expandable chamber 1106 and the second expandable chamber 1116 are properly inflated.

As illustrated in FIG. 11, the nucleus implant 1100 includes a second injection tube 1122 that extends from the outer surface 1120 of the second expandable chamber 1116. In a particular embodiment, the second expandable chamber 1116 of the nucleus implant 1100 is expandable from a deflated position, shown in FIG. 11, to one selected position among a plurality of inflated positions, shown in FIG. 12, up to a maximum inflated position. Further, after the second expandable chamber 1116 is inflated, or otherwise expanded, the second injection tube 1122 can be removed, as depicted in FIG. 12.

Additionally, the nucleus implant 1100 can include a core holder 1124 that extends from the surface of the solid core 1102. The core holder 1124 can be used to position the nucleus implant 1100 and hold the nucleus implant 1100 in the proper position while the first expandable chamber 1106 and the second expandable chamber 1116 are inflated. Moreover, the core holder 1124 can be removed after the first expandable chamber 1106 and the second expandable chamber 1116 are inflated. An implant with several chambers surrounding a core may enable more fine adjustment of the position of a core than with a single chamber.

In a particular embodiment, the nucleus implant 1100 can include a first self-sealing valve (not shown) within the outer surface 1110 of the first expandable chamber 1106, e.g., adjacent to the first injection tube 1112. Further, the nucleus implant 1100 can include a second self-sealing valve (not shown) within the outer surface 1120 of the second expandable chamber 1116, e.g., adjacent to the second injection tube 1122. The self-sealing valves can prevent the expandable chambers 1106, 1116 from leaking material after the expandable chambers 1106, 1116 are inflated and the injection tubes 1112, 1122 are removed.

FIG. 13 indicate that the nucleus implant 1100 can be implanted within an intervertebral disc 1200 between a superior vertebra 1300 and an inferior vertebra 1302. More specifically, the nucleus implant 1100 can be implanted within an intervertebral disc space 1202 established within the annulus fibrosus 1204 of the intervertebral disc 1200. The intervertebral disc space 1202 can be established by removing the nucleus pulposus (not shown) from within the annulus fibrosus 1204.

In a particular embodiment, the first expandable chamber 1106 can be inflated so the inner surface 1108 of the first expandable chamber 1106 engages the outer surface of the solid core 1102 and the outer surface 1110 of the first expandable chamber 1106 engages the inner surface 1118 of the second expandable chamber 1116. Further, the outer surface 1120 of the second expandable chamber 1116 can engage the annulus fibrosis 1204.

The nucleus implant 1100 can provide shock-absorbing characteristics substantially similar to the shock absorbing characteristics provided by the nucleus pulposus. Further, in a particular embodiment, the hardness of the solid core 1102 of the nucleus implant 1100 is greater than or equal to the hardness of the material used to inflate the first expandable chamber 1106, i.e., after that material is cured. Further, the hardness of the material used to inflate the first expandable chamber 1106 is greater than or equal to the hardness of the material used to inflate the second expandable chamber 1116, e.g., after those materials cure.

Arranging several expandable chambers around a core may result in an implant with a hardness that varies more progressively from the core towards the periphery than with a single chamber. Thus, an implant with a very hard core and a very soft periphery may be obtained. Moreover, an implant with several variable hardness chambers may more easily spread the loads exerted at the vertebral level. In addition, the mobility of such an arranged implant may be better controlled. In one example, the core has a hardness of 55 Shore D, the first chamber has a hardness of 50 Shore D and the second chamber has a hardness of 40 Shore D.

Arranging several expandable chambers around a core enables to obtain an implant, the hardness of which varies more progressively from the core towards the periphery than with a single chamber. Thus, an implant with a very hard core and a very soft periphery may be obtained. Moreover, an implant with several variable hardness chambers enables to more easily spread the loads exerted at the level of the vertebras. In addition, the mobility of the thus arranged implant is better controlled. By way of example, the core has a hardness of 55 Shore D, the first chamber has a hardness of 50 Shore D and the second chamber has a hardness of 40 Shore D

Additionally, the height of the solid core 1102 can be greater than or equal to the height of the first expandable chamber 1106 when fully expanded. Also, the height of the first expandable chamber 1106 when fully expanded can be greater than or equal to the height of the second expandable chamber 1116 when fully expanded. As shown in FIG. 13, the solid core 1102, the first expandable chamber 1106, and the second expandable chamber 1116 of the nucleus implant 1100 can be configured to provide proper support and spacing between the superior vertebra 1300 and the inferior vertebra 1302.

In a particular embodiment, the first expandable chamber 1106, the second expandable chamber 1116, or a combination of the first expandable chamber 1106 and the second expandable chamber 1116 of the nucleus implant 1100 can be inflated with one or more injectable extended use approved medical materials that remain elastic after curing. It will be appreciated that the material or materials used for injection can be different for the two chambers. Further, the injectable extended use approved medical materials can include polymer materials that remain elastic after curing.

For example, the polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Also, the silicone materials can include a silicone hydrogel.

In an alternative embodiment, the injectable extended use approved medical materials can include one or more fluids such as sterile water, saline, or sterile air. In alternative embodiments, the first expandable chamber 1106, the second expandable chamber 1116, or a combination of the first expandable chamber 1106 and the second expandable chamber 1116 of the nucleus implant 1100 can be inflated with one or more of the following: fibroblasts, lipoblasts, chondroblasts, differentiated stem cells or other biologic factor which would create a motion limiting tissue when injected into a bioresorbable motion limiting scaffold.

In a particular embodiment, the nucleus implant 1100 can be installed using a posterior surgical approach, as shown. Further, the nucleus implant 1100 can be installed through a posterior incision 1206 made within the annulus fibrosus 1204 of the intervertebral disc 1200. Alternatively, the nucleus implant 1100 can be installed using an anterior surgical approach, a lateral surgical approach, or any other surgical approach well known in the art.

### Description of a Fourth Embodiment of a Nucleus Implant

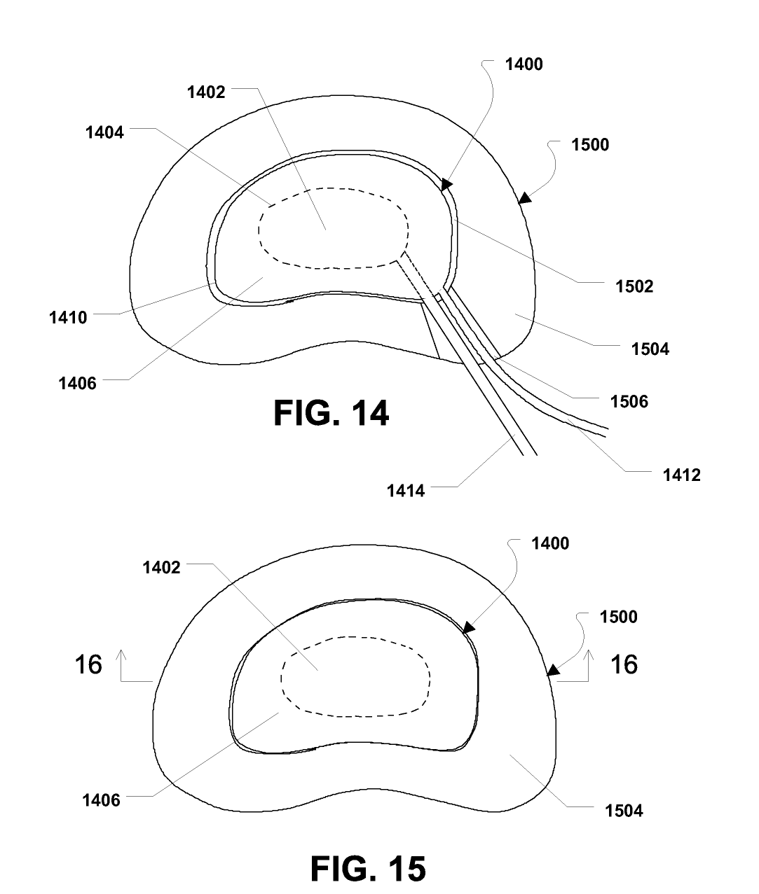

Referring to FIG. 14 through FIG. 16, an embodiment of a nucleus implant is shown and is designated 1400. As shown, the nucleus implant 1400 includes a solid core 1402 that defines an outer surface 1404. In a particular embodiment, the solid core 1402 can have a cross-section that is generally elliptical. Alternatively, the solid core 1402 can have a cross-section that is: generally circular, generally rectangular, generally square, generally triangular, generally trapezoidal, generally rhombic, generally quadrilateral, any generally polygonal shape, or any combination thereof.

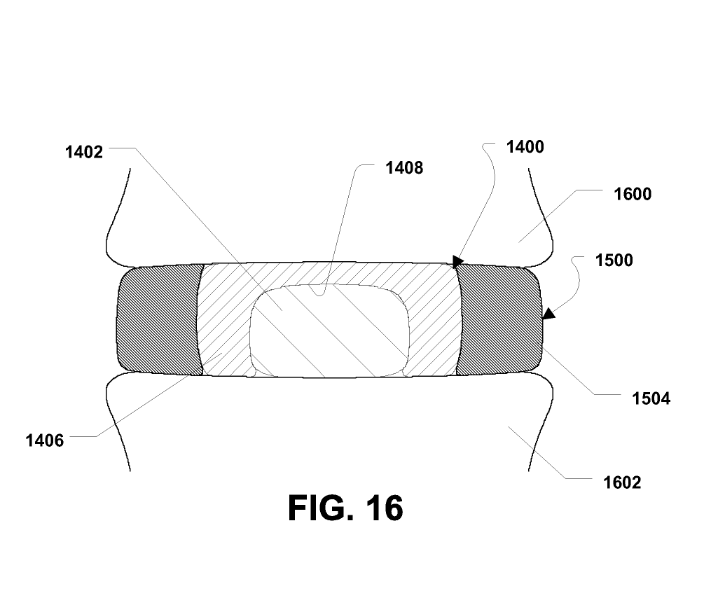

As illustrated in FIG. 14 through FIG. 16, an expandable chamber 1406 can be disposed around the solid core 1402. In a particular embodiment, as shown, the expandable chamber 1406 can have a generally inverted-bowl shape and the expandable chamber 1406 can be draped, or otherwise placed, over the solid core 1402 and inflated or expanded, as shown in FIG. 16.

The thus shaped chamber that is arranged around the core may enable accurate positioning of the core. The accuracy of the core positioning may be increased by inflating the chamber with a uniform or substantially uniform pressure.

The expandable chamber 1406 can define an inner surface 1408 and an outer surface 1410. In a particular embodiment, the inner surface 1408 of the expandable chamber 1406 can be attached to the outer surface 1404 of the solid core 1402. As such, proper placement of the expandable chamber 1406 can be based on the placement of the solid core 1402. Alternatively, the expandable chamber 1406 can be separate from the solid core 1402 and the expandable chamber 1406 may engage the solid core 1402 after the expandable chamber 1406 is properly inflated.

As depicted in FIG. 14, the nucleus implant 1400 can include an injection tube 1412 that extends from the outer surface 1410 of the expandable chamber 1406. In a particular embodiment, the expandable chamber 1406 of the nucleus implant 1400 is expandable from a deflated position, shown in FIG. 14, to one selected position among a plurality of inflated positions, shown in FIG. 15, up to a maximum inflated position. Further, after the expandable chamber 1406 is inflated, or otherwise expanded, the injection tube 1412 can be removed, as depicted in FIG. 15.

Additionally, the nucleus implant 1400 can include a core holder 1414 that extends from the surface of the solid core 1402. The core holder 1414 can be used to position the nucleus implant 1400 and hold the nucleus implant 1400 in the proper position while the expandable chamber 1406 is inflated. Moreover, the core holder 1414 can be removed after the expandable chamber 1406 is inflated. In a particular embodiment, the nucleus implant 1400 can include a self-sealing valve (not shown) within the outer surface 1410 of the expandable chamber 1406, e.g., adjacent to the injection tube 1412. The self-sealing valve can prevent the expandable chamber 1406 from leaking material after the expandable chamber 1406 is inflated and the injection tube 1412 is removed.

FIG. 16 indicates that the nucleus implant 1400 can be implanted within an intervertebral disc 1500 between a superior vertebra 1600 and an inferior vertebra 1602. More specifically, the nucleus implant 1400 can be implanted within an intervertebral disc space 1502 established within the annulus fibrosus 1504 of the intervertebral disc 1500. The intervertebral disc space 1502 can be established by removing the nucleus pulposus (not shown) from within the annulus fibrosus 1504.

In a particular embodiment, the expandable chamber 1406 can be inflated so the inner surface 1408 of the expandable chamber 1406 engages the outer surface of the solid core 1402 and the outer surface 1410 of the expandable chamber 1406 engages the annulus fibrosis 1504. Further, portions of the outer surface 1410 of the expandable chamber 1406 can engage the superior vertebra 1600 and an inferior vertebra 1602. Moreover, when the expandable chamber 1406 is expanded, or otherwise inflated, a portion of the expandable chamber 1406 is located between the solid core 1402 and the superior vertebra 1600.

It will be appreciated that in a particular embodiment, the arrangements of the implant of FIGS. 13 and 16 may be assembled within the same implant. Thus, for example, the inverted-bowl shaped chamber 1406 may be formed of a first chamber having an inverted-bowl shape and a second peripheral chamber similar to the chamber 1116 of FIG. 13 and that peripherally surrounds this first chamber. The advantages related to each of both implants of FIGS. 13 and 16 may thus be obtained with a single implant.

The nucleus implant 1400 can provide shock-absorbing characteristics substantially similar to the shock absorbing characteristics provided by the nucleus pulposus. Further, in a particular embodiment, the hardness of the solid core 1402 of the nucleus implant 1400 is greater than or equal to the hardness of the material used to inflate the expandable chamber 1406, i.e., after that material is cured. As shown in FIG. 16, the solid core 1402 and the expandable chamber 1406 of the nucleus implant 1400 can be configured to provide proper support and spacing between the superior vertebra 1600 and the inferior vertebra 1602.

In a particular embodiment, the expandable chamber 1406 of the nucleus implant 1400 can be inflated with one or more injectable extended use approved medical materials that remain elastic after curing. Further, the injectable extended use approved medical materials can include polymer materials that remain elastic after curing.

For example, the polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Also, the silicone materials can include a silicone hydrogel.

In an alternative embodiment, the injectable extended use approved medical materials can include one or more fluids such as sterile water, saline, or sterile air. In alternative embodiments, the expandable chamber 1406 of the nucleus implant 1400 can be inflated with one or more of the following: fibroblasts, lipoblasts, chondroblasts, differentiated stem cells or other biologic factor which would create a motion limiting tissue when injected into a bioresorbable motion limiting scaffold.

In a particular embodiment, the nucleus implant 1400 can be installed using a posterior surgical approach, as shown. Further, the nucleus implant 1400 can be installed through a posterior incision 1506 made within the annulus fibrosus 1504 of the intervertebral disc 1500. Alternatively, the nucleus implant 1400 can be installed using an anterior surgical approach, a lateral surgical approach, or any other surgical approach well known in the art.

### Description of a Fifth Embodiment of a Nucleus Implant

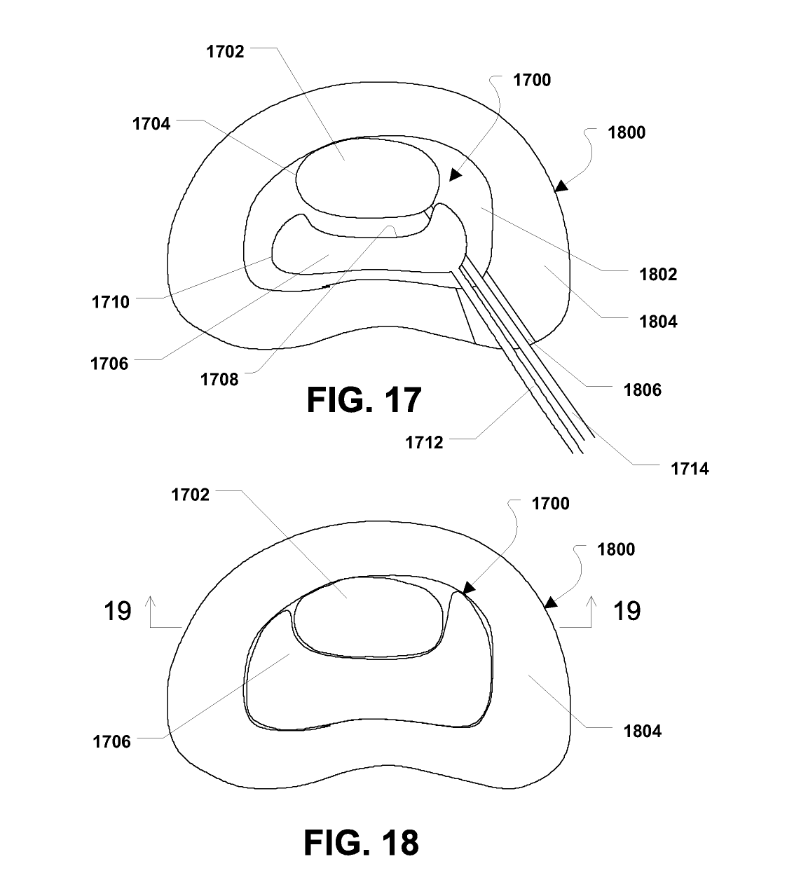

Referring to FIG. 17 through FIG. 19, an embodiment of a nucleus implant is shown and is designated 1700. As shown, the nucleus implant 1700 includes a solid core 1702 that defines an outer surface 1704. In a particular embodiment, the solid core 1702 can have a cross-section that is generally elliptical. Alternatively, the solid core 1702 can have a cross-section that is: generally circular, generally rectangular, generally square, generally triangular, generally trapezoidal, generally rhombic, generally quadrilateral, any generally polygonal shape, or any combination thereof.

As illustrated in FIG. 17 through FIG. 19, an expandable chamber 1706 can be disposed around the solid core 1702. In a particular embodiment, as shown, the expandable chamber 1706 can be generally shaped like the letter “U” and the expandable chamber 1706 can be inflated, or otherwise expanded, around the solid core 1702.

The U-shaped chamber may be particularly suited for avoiding the migration of the core towards the incision through which it has been inserted. This is because the U-shape partially surrounding the core conceals this incision. This U-shape is also advantageous when the intervertebral disc shape has, in a sagittal plane, an obvious trapezoidal shape. A U-shape enables the chamber to suitably occupy the space on three sides of the core. It is to be noted that an intermediate expandable chamber occupying the space between the core 1702 and the U chamber 1706 (FIG. 17) may be envisaged. This additional arrangement may result in more accurate positioning of the core.

The expandable chamber 1706 can define a first surface 1708 and a second surface 1710. In a particular embodiment, the first surface 1708 of the expandable chamber 1706 can be attached to the outer surface 1704 of the solid core 1702. As such, proper placement of the expandable chamber 1706 can be based on the placement of the solid core 1702. Alternatively, the expandable chamber 1706 can be separate from the solid core 1702 and the expandable chamber 1706 may engage the solid core 1702 after the expandable chamber 1706 is properly inflated.

As depicted in FIG. 17, the nucleus implant 1700 can include an injection tube 1712 that extends from the second surface 1710 of the expandable chamber 1706. In a particular embodiment, the expandable chamber 1706 of the nucleus implant 1700 is expandable from a deflated position, shown in FIG. 17, to one selected position among a plurality of inflated positions, shown in FIG. 18, up to a maximum inflated position. Further, after the expandable chamber 1706 is inflated, or otherwise expanded, the injection tube 1712 can be removed, as depicted in FIG. 18.

Additionally, the nucleus implant 1700 can include a core holder 1714 that extends from the surface of the solid core 1702. The core holder 1714 can be used to position the nucleus implant 1700 and hold the nucleus implant 1700 in the proper position while the expandable chamber 1706 is inflated. Moreover, the core holder 1714 can be removed after the expandable chamber 1706 is inflated. In a particular embodiment, the nucleus implant 1700 can include a self-sealing valve (not shown) within the second surface 1710 of the expandable chamber 1706, e.g., adjacent to the injection tube 1712. The self-sealing valve can prevent the expandable chamber 1706 from leaking material after the expandable chamber 1706 is inflated and the injection tube 1712 is removed.

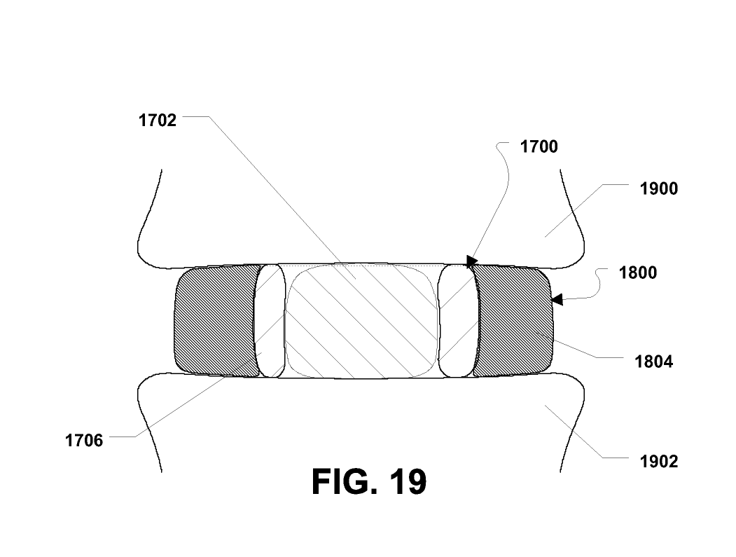

FIG. 19 indicates that the nucleus implant 1700 can be implanted within an intervertebral disc 1800 between a superior vertebra 1900 and an inferior vertebra 1902. More specifically, the nucleus implant 1700 can be implanted within an intervertebral disc space 1802 established within the annulus fibrosus 1804 of the intervertebral disc 1800. The intervertebral disc space 1802 can be established by removing the nucleus pulposus (not shown) from within the annulus fibrosus 1804.

In a particular embodiment, the expandable chamber 1706 can be inflated so the first surface 1708 of the expandable chamber 1706 engages a portion of the outer surface of the solid core 1702 and the second surface 1710 of the expandable chamber 1706 engages a portion of the annulus fibrosis 1804. Further, portions of the outer surface 1710 of the expandable chamber 1706 can engage the superior vertebra 1900 and an inferior vertebra 1902. Moreover, when the expandable chamber 1706 is expanded, or otherwise inflated, the expandable chamber 1706 at least partially surrounds the solid core 1702. As depicted in FIG. 18, the core 1702 may be placed between the arms of the U formed by the chamber 1706.

The nucleus implant 1700 can provide shock-absorbing characteristics substantially similar to the shock absorbing characteristics provided by the nucleus pulposus. Further, in a particular embodiment, the hardness of the solid core 1702 of the nucleus implant 1700 is greater than or equal to the hardness of the material used to inflate the expandable chamber 1706, i.e., after that material is cured. Also, the overall height of the solid core 1702 can be greater than or equal to the overall height of the expandable chamber 1706 when inflated. As shown in FIG. 19, the solid core 1702 and the expandable chamber 1706 of the nucleus implant 1700 can be configured to provide proper support and spacing between the superior vertebra 1900 and the inferior vertebra 1902.

In a particular embodiment, the expandable chamber 1706 of the nucleus implant 1700 can be inflated with one or more injectable extended use approved medical materials that remain elastic after curing. Further, the injectable extended use approved medical materials can include polymer materials that remain elastic after curing.

For example, the polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Also, the silicone materials can include a silicone hydrogel.

In an alternative embodiment, the injectable extended use approved medical materials can include one or more fluids such as sterile water, saline, or sterile air. In alternative embodiments, the expandable chamber 1706 of the nucleus implant 1700 can be inflated with one or more of the following: fibroblasts, lipoblasts, chondroblasts, differentiated stem cells or other biologic factor which would create a motion limiting tissue when injected into a bioresorbable motion limiting scaffold.

In a particular embodiment, the nucleus implant 1700 can be installed using a posterior surgical approach, as shown. Further, the nucleus implant 1700 can be installed through a posterior incision 1806 made within the annulus fibrosus 1804 of the intervertebral disc 1800. Alternatively, the nucleus implant 1700 can be installed using an anterior surgical approach, a lateral surgical approach, or any other surgical approach well known in the art.

### Description of an Embodiment of a Method of Installing a Nucleus Implant

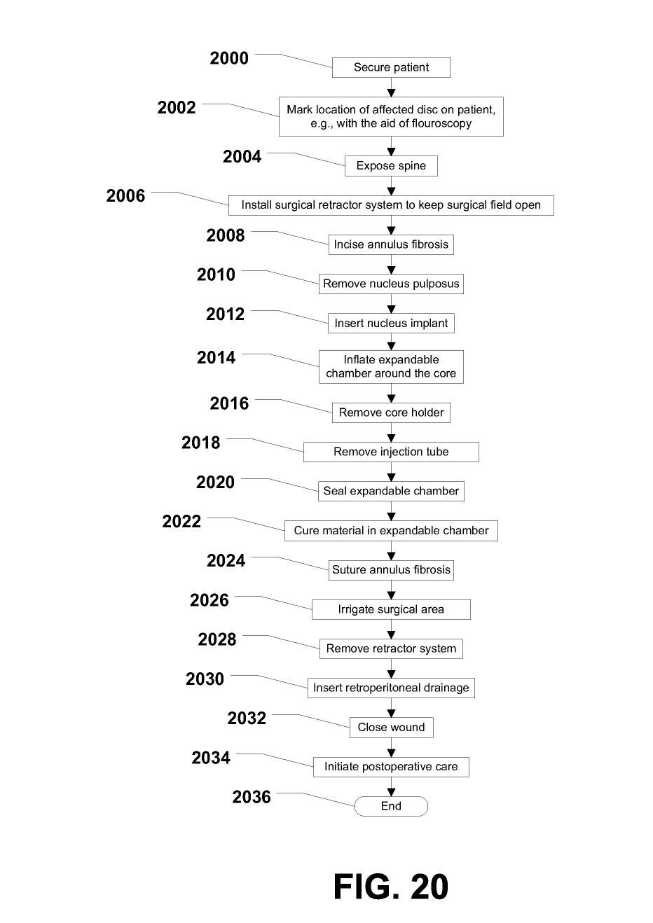

Referring to FIG. 20, an exemplary, non-limiting embodiment of a method of installing a nucleus implant is shown and commences at block 2000. At block 2000, a patient is secured on an operating table. For example, the patient can be secured in a supine position to allow an anterior approach to be used to access the patient's spinal column. Further, the patient may be placed in a “French” position in which the patient's legs are spread apart. The “French” position can allow the surgeon to stand between the patient's legs. Further, the “French” position can facilitate proper alignment of the surgical instruments with the patient's spine. In another particular embodiment, the patient can be secured in the supine position on an adjustable surgical table.

In one or more alternative embodiments, a surgeon can use a posterior approach or a lateral approach to implant an intervertebral prosthetic device. As such, the patient may be secured in a different position, e.g., in a prone position for a posterior approach or in a lateral decubitus position for a lateral approach.

Moving to block 2002, the location of the affected disc is marked on the patient, e.g., with the aid of fluoroscopy. At block 2004, the surgical area along spinal column is exposed. Further, at block 2006, a surgical retractor system can be installed to keep the surgical field open. For example, the surgical retractor system can be a Medtronic Sofamor Danek Endoring™ Surgical Retractor System.

Proceeding to block 2008, the annulus fibrosus of the affected disc is incised to expose the nucleus pulposus. Further, at block 2010, the nucleus pulposus is removed to create an intervertebral disc space within the annulus fibrosus. At block 2012, the nucleus implant is inserted within the intervertebral disc space of the annulus fibrosus. Further, at block 2014, the expandable chamber is inflated, or otherwise expanded, around the core, thereby positioning and retaining the core. At block 2016, the core holder is removed. Further, at block 2018, the injection tube can be removed.

Continuing to block 2020, the expandable chamber is sealed—if the expandable chamber is not self-sealing, e.g., with a self-sealing valve. At block 2022, the material used to inflate, or expand, the expandable chamber can be cured. In a particular embodiment, the material can be allowed to cure naturally under the ambient conditions of the operating room. Alternatively, the material can be cured using an energy source. For example, the energy source can be a light source that emits visible light, infrared (IR) light, or ultra-violet (UV) light. Further, the energy source can be a heating device, a radiation device, or other mechanical device.

Proceeding to block 2024, the annulus fibrosus is sutured. At block 2026, the intervertebral space can be irrigated. Further, at block 2028, the retractor system can be removed. At block 2030, a drainage, e.g., a retroperitoneal drainage, can be inserted into the wound. Additionally, at block 2032, the surgical wound can be closed. The surgical wound can be closed using sutures, surgical staples, or any other surgical technique well known in the art. Moving to block 2034, postoperative care can be initiated. The method ends at state 2036. Conclusion

With the configuration of structure described above, the nucleus implant according to one or more of the embodiments provides a device that may be implanted to replace the nucleus pulposus within a natural intervertebral disc that is diseased, degenerated, or otherwise damaged. The nucleus implant can be disposed within an intervertebral disc space that can be established within an intervertebral disc by removing the nucleus pulposus.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A nucleus implant configured to be installed within an intervertebral disc between an inferior vertebra and a superior vertebra, the nucleus implant comprising:
a solid core; and
an expandable chamber disposed at least partially around the solid core, wherein the expandable chamber is expandable from a deflated position to an inflated position.

2. The nucleus implant of claim 1, wherein the expandable chamber is configured to engage an annulus fibrosus when inflated.

3. The nucleus implant of claim 1, wherein a hardness of the solid core is greater than or equal to a hardness of the expandable chamber when the expandable chamber is inflated.

4. The nucleus implant of claim 1, wherein a height of the solid core is greater than or equal to a height of the expandable chamber when the expandable chamber is inflated.

5. The nucleus implant of claim 2, wherein the expandable chamber is inflated with an injectable extended use approved medical material.

6. The nucleus implant of claim 5, wherein the injectable extended use approved medical material comprises a polymer material.

7. The nucleus implant of claim 6, wherein the polymer material comprises a polyurethane material, a polyolefin material, a polyether material, a silicone material, or a combination thereof.

8. The nucleus implant of claim 7, wherein the polyolefin material comprises a polypropylene, polyethylene, halogenated polyolefin, flouropolyolefin, or a combination thereof.

9. The nucleus implant of claim 7, wherein the polyether material comprises polyetherketone (PEK), polyetheretherketone (PEEK), polyetherketoneketone (PEKK), polyaryletherketone (PAEK), or a combination thereof.

10. The nucleus implant of claim 7, wherein the silicon material comprises silicone hydrogel.

11. The nucleus implant of claim 5, wherein the injectable extended use approved medical material comprises sterile water, saline, sterile air, or a combination thereof.

12. The nucleus implant of claim 1, further comprising a core holder extending from the solid core wherein the core holder is configured to be removed after the nucleus implant is installed within an intervertebral disc.

13. The nucleus implant of claim 12, further comprising an injection tube extending from the expandable chamber, wherein the injection tube is configured to be removed after the nucleus implant is installed within an intervertebral disc.

14. The nucleus implant of claim 1, wherein the expandable chamber is generally toroid shaped and includes an inner surface and wherein the inner surface of the expandable chamber is configured to engage an outer surface of the solid core when the expandable chamber is inflated.

15. The nucleus implant of claim 14, wherein the inner surface of the expandable chamber is attached to the outer surface of the solid core.

16. The nucleus implant of claim 1, wherein the solid core is formed with a hole.

17. The nucleus implant of claim 1, wherein the expandable chamber is a first expandable chamber and wherein the nucleus implant further comprises a second expandable chamber at least partially around the first expandable chamber.

18. The nucleus implant of claim 17, wherein the second expandable chamber is expandable from a deflated position to one inflated position.

19. The nucleus implant of claim 18, wherein the second expandable chamber is configured to engage an annulus fibrosus when inflated.

20. The nucleus implant of claim 1, wherein the expandable chamber is generally shaped like an inverted bowl and wherein the expandable chamber is installed over the solid core.

21. The nucleus implant of claim 1, wherein the expandable chamber is configured to engage an annulus fibrosis, the superior vertebra, the inferior vertebra, or a combination thereof.

22. The nucleus implant of claim 21, wherein a portion of the expandable chamber is disposed between the solid core and a vertebra.

23. The nucleus implant of claim 1, wherein the expandable chamber is generally shaped like the letter U.

24. The nucleus implant of claim 23, wherein the expandable chamber is configured to be inflated around the solid core.

25. The nucleus implant of claim 24, wherein the solid core is configured to be installed in an anterior position within an intervertebral disc and wherein the expandable chamber is configured to be installed posterior to the solid core.

26. A nucleus implant configured to be installed within an intervertebral disc between an inferior vertebra and a superior vertebra, the nucleus implant comprising:
a solid core including an outer surface; and
a toroid shaped expandable chamber disposed at least partially around the solid core, wherein the toroid shaped expandable chamber includes an inner surface and an outer surface, and wherein the inner surface of the toroid shaped expandable chamber is configured to engage the outer surface of the solid core and wherein the outer surface of the toroid shaped expandable chamber is configured to engage an annulus fibrosus of an intervertebral disc.

27. The nucleus implant of claim 26, wherein the toroid shaped expandable chamber is expandable from a deflated position to an inflated position.

28. The nucleus implant of claim 27, further comprising a core holder extending from the solid core wherein the core holder is configured to assist in positioning the nucleus implant within the intervertebral disc and wherein the core holder is configured to be removed after the nucleus implant is installed.

29. The nucleus implant of claim 28, further comprising an injection tube extending from the toroid shaped expandable chamber, wherein the injection tube is configured to be removed after the toroid shaped expandable chamber is inflated.

30. The nucleus implant of claim 26, wherein the solid core is formed with a hole.

31. The nucleus implant of claim 26, wherein a hardness of the solid core is greater than or equal to a hardness of the toroid shaped expandable chamber when the toroid shaped expandable chamber is inflated.

32. The nucleus implant of claim 26, wherein a height of the solid core is greater than or equal to a height of the toroid shaped expandable chamber when the toroid shaped expandable chamber is inflated.

33. The nucleus implant of claim 26, wherein the toroid shaped expandable chamber is attached to the solid core.

34. The nucleus implant of claim 33, wherein the inner surface of the expandable chamber is attached to the outer surface of the solid core.

35. A nucleus implant configured to be installed within an intervertebral disc between an inferior vertebra and a superior vertebra, the nucleus implant comprising:
a solid core including an outer surface;
a first toroid shaped expandable chamber disposed at least partially around the solid core, wherein the first toroid shaped expandable chamber includes an inner surface and an outer surface, and wherein the inner surface of the first toroid shaped expandable chamber is configured to engage the outer surface of the solid core; and
a second toroid shaped expandable chamber disposed at least partially around the first toroid shaped expandable chamber, wherein the second toroid shaped expandable chamber includes an inner surface and an outer surface, wherein the inner surface of the second expandable chamber is configured to engage the outer surface of the first toroid shaped expandable chamber, and wherein the outer surface of the second toroid shaped expandable chamber is configured to engage an annulus fibrosus of the intervertebral disc.

36. The nucleus implant of claim 35, wherein the first toroid shaped expandable chamber is expandable from a deflated position to an inflated position and wherein the second toroid shaped expandable chamber is expandable from a deflated position to an inflated position.

37. The nucleus implant of claim 36, further comprising a core holder extending from the solid core wherein the core holder is configured to assist in positioning the nucleus implant within the intervertebral disc and wherein the core holder is configured to be removed after the nucleus implant is installed.

38. The nucleus implant of claim 37, further comprising a first injection tube extending from the first toroid shaped expandable chamber, wherein the first injection tube is configured to be removed after the first toroid shaped expandable chamber is inflated.

39. The nucleus implant of claim 38, further comprising a second injection tube extending from the second toroid shaped expandable chamber, wherein the second injection tube is configured to be removed after the second toroid shaped expandable chamber is inflated.

40. The nucleus implant of claim 35, wherein the solid core is formed with a hole.

41. The nucleus implant of claim 35, wherein a hardness of the solid core is greater than or equal to a hardness of the first toroid shaped expandable chamber when the first toroid shaped expandable chamber is inflated and the hardness of the first toroid shaped expandable chamber is greater than or equal to a hardness of the second toroid shaped expandable chamber when the first toroid shaped expandable chamber and the second toroid shaped expandable chamber are inflated.

42. The nucleus implant of claim 35, wherein a height of the solid core is greater than or equal to a height of the first expandable chamber when the first toroid shaped expandable chamber is inflated and wherein the height of the first toroid shaped expandable chamber is greater than or equal to a height of the second toroid shaped expandable chamber when the first toroid shaped expandable chamber and the second toroid shaped expandable chamber are inflated.

43. The nucleus implant of claim 35, wherein the first toroid shaped expandable chamber is attached to the solid core and wherein the second toroid shaped expandable chamber is attached to the first toroid shaped expandable chamber.

44. The nucleus implant of claim 43, wherein the inner surface of the first toroid shaped expandable chamber is attached to the outer surface of the solid core and wherein the inner surface of the second toroid shaped expandable chamber is attached to the outer surface of the second toroid shaped expandable chamber.

45. A nucleus implant configured to be installed within an intervertebral disc between an inferior vertebra and a superior vertebra, the nucleus implant comprising:
a solid core including an outer surface; and
a bowl shaped expandable chamber disposed at least partially around the solid core, wherein the bowl shaped expandable chamber includes an inner surface and an outer surface, and wherein the inner surface of the bowl shaped expandable chamber is configured to engage the outer surface of the solid core and wherein the outer surface of the bowl shaped expandable chamber is configured to engage an annulus fibrosus of the intervertebral disc, the superior vertebra, the inferior vertebra, or a combination thereof.

43. The nucleus implant of claim 42, wherein the bowl shaped expandable chamber is expandable from a deflated position to an inflated position.

44. The nucleus implant of claim 42, further comprising a core holder extending from the solid core wherein the core holder is configured to assist in positioning the nucleus implant within the intervertebral disc and wherein the core holder is configured to be removed after the nucleus implant is installed.

45. The nucleus implant of claim 44, further comprising an injection tube extending from the bowl shaped expandable chamber, wherein the injection tube is configured to be removed after the bowl shaped expandable chamber is inflated.

46. The nucleus implant of claim 42, wherein a hardness of the solid core is greater than or equal to a hardness of the bowl shaped expandable chamber when the bowl shaped expandable chamber is inflated.

47. The nucleus implant of claim 42, wherein the bowl shaped expandable chamber is attached to the solid core.

48. The nucleus implant of claim 47, wherein the inner surface of the bowl shaped expandable chamber is attached to the outer surface of the solid core.

49. A nucleus implant configured to be installed within an intervertebral disc between an inferior vertebra and a superior vertebra, the nucleus implant comprising:
a solid core including an outer surface; and
a U shaped expandable chamber disposed at least partially around the solid core, wherein the U shaped expandable chamber includes a first surface and a second surface, and wherein the first surface of the U shaped expandable chamber is configured to engage the outer surface of the solid core and wherein the second surface of the U shaped expandable chamber is configured to engage an annulus fibrosus of the intervertebral disc.

50. The nucleus implant of claim 49, wherein the U shaped expandable chamber is expandable from a deflated position to an inflated position.

51. The nucleus implant of claim 50, further comprising a core holder extending from the solid core wherein the core holder is configured to assist in positioning the nucleus implant within the intervertebral disc and wherein the core holder is configured to be removed after the nucleus implant is installed.

52. The nucleus implant of claim 51, further comprising an injection tube extending from the U shaped expandable chamber, wherein the injection tube is configured to be removed after the U shaped expandable chamber is inflated.

53. The nucleus implant of claim 49, wherein a hardness of the solid core is greater than or equal to a hardness of the U shaped expandable chamber when the U shaped expandable chamber is inflated.

54. The nucleus implant of claim 49, wherein a height of the solid core is greater than or equal to a height of the U shaped expandable chamber when the U shaped expandable chamber is inflated.

55. The nucleus implant of claim 49, wherein the U shaped expandable chamber is attached to the solid core.

56. The nucleus implant of claim 55, wherein the first surface of the U shaped expandable chamber is attached to the outer surface of the solid core.

57. The nucleus implant of claim 49, wherein the solid core is configured to be installed in an anterior position within the intervertebral disc and wherein the U shaped expandable chamber is configured to be installed at least partially posterior to the solid core.

58. A method of installing a nucleus implant within an intervertebral disc between an inferior vertebra and a superior vertebra of a patient, the method comprising:
implanting the nucleus implant within the intervertebral disc, wherein the nucleus implant includes a solid core and an expandable chamber at least partially around the solid core; and
inflating the expandable chamber around the solid core, wherein the expandable chamber includes an outer surface configured to engage an annulus fibrosus of the intervertebral disc when the expandable chamber is inflated and wherein a hardness of the solid core is greater than or equal to a hardness of the expandable chamber.

59. The method of claim 58, further comprising removing a core holder from the solid core.

60. The method of claim 59, further comprising removing an injection tube from the expandable chamber.

61. The method of claim 60, further comprising sealing the expandable chamber.

62. The method of claim 60, further comprising curing a material used to inflate the expandable chamber.

63. The method of claim 58, wherein the expandable chamber is inflated using an injectable extended use approved medical material.

64. The method of claim 63, wherein the injectable extended use approved medical material is a polymer material.

65. The method of claim 64, wherein the polymer material is a polyurethane material, a polyolefin material, a polyether material, a silicone material, or a combination thereof.

66. The method of claim 65, wherein the polyolefin material is a polypropylene, polyethylene, halogenated polyolefin, flouropolyolefin, or a combination thereof.

67. The method of claim 65, wherein the polyether material is polyetherketone (PEK), polyetheretherketone (PEEK), polyetherketoneketone (PEKK), polyaryletherketone (PAEK), or a combination thereof.

68. The method of claim 65, wherein the silicon material is silicone hydrogel.

69. The method of claim 63, wherein the injectable extended use approved medical material comprises sterile water, saline, sterile air, or a combination thereof.
